(19) United States
(12) Patent Application Publication
(10) Pub. No.: US 2013/0253587 A1
(43) Pub. Date: Sep. 26, 2013

(54) SPINAL SYSTEMS AND METHODS FOR CORRECTION OF SPINAL DISORDERS

(71) Applicants: Thomas A. Carls, Memphis, TN (US); Newton Metcalf, Memphis, TN (US)

(72) Inventors: Thomas A. Carls, Memphis, TN (US); Newton Metcalf, Memphis, TN (US)

(73) Assignee: WARSAW ORTHOPEDIC, INC., Memphis, TN (US)

(21) Appl. No.: 13/425,057

(22) Filed: Mar. 20, 2012

(51) Int. Cl. A61B 17/70; A61B 17/88

(52) U.S. Cl. 606/263; 606/246; 606/278; 606/279

(57) ABSTRACT

A system for reducing curvature of a spine is provided, the system comprising a spinal construct having an elongated longitudinal element affixed to and extending between a first fixation element and a second fixation element, the first fixation element having a first end configured to engage at least a portion of a first anchor member, and the second fixation element having a second end configured to engage at least a portion of a second anchor member, the first and second anchor members configured to pierce the spine, wherein the elongated longitudinal element is configured to generate a corrective force sufficient to reduce curvature of the spine. The systems and methods provided allows a surgeon to select a tether, determine its length, and pre-assemble the spinal construct, which then can be coupled onto the head of a bone anchor.

### FIELD

The present disclosure generally relates to medical devices for the treatment of musculoskeletal disorders, and more particularly to a spinal construct for fusionless correction of a spine disorder.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Normally, the spinal column grows in line from the neck to the tailbone and, when viewed from the side, curves are seen in the neck, upper trunk, and lower trunk. The upper trunk has a gentle rounded contour called kyphosis and the lower trunk has a reverse of the rounded contour called lordosis. Certain amounts of cervical (neck) lordosis, thoracic (upper back) kyphosis, and lumbar (lower back) lordosis are normally present and are needed to maintain appropriate trunk balance over the pelvis. Deviations from this normal alignment may reflect abnormal kyphosis or lordosis when viewed from the side, or more commonly, scoliosis, when viewed from the anterior or posterior.

Under normal circumstances major joints contain one or more articular junctions occurring between bony structures and several soft tissue (ligamentous and tendonous) attachments that are integral to motion and stability of the joint structure. Compromise of these soft tissue attachments results in partial to complete loss of joint function and stability.

Scoliosis is a sequential misalignment or deformity of the bones and discs of the spine and is manifested in the following ways. First, the deformity can be an apparent side bending of the spine when viewed in a coronal plane from the front or back (anterior/posterior or AP view). Second, another way of diagnosing scoliosis is a loss of the normal kyphotic curvature in the thoracic or chest area when viewed from the side. This is a sagittal plane deformity. And third, scoliosis can be observed as a result of the rotation of the spine around its own long axis. This is an axial plane deformity. If scoliosis is left untreated, the curve can progress and eventually cause pain, significant cosmetic deformity, and heart, lung, or gastrointestinal problems.

Soft tissue damage leads to the loss of function, stability or alignment of the major articular joint structures and is diagnosed in the following manner. First, physical examination of the joint and its motion characteristics may be performed to determine the extent of the loss of function and stability. Second, arthroscopic or radiographic, particularly MRI, methods may be used to further refine the physical diagnosis. Depending on the extent of the injury, some patients may function at an acceptable level without surgical intervention while others require major reconstruction to function reasonably well.

The ultimate goal of treatment for scoliosis is the creation of desirable curvature in a portion of the spine. Some cases of scoliosis, if diagnosed at its earlier stages, can be managed without surgery. Otherwise, the curvature should be corrected by surgical procedures. Typically, a surgical procedure is associated with stainless steel or titanium rods affixed to the bone with hooks or screws, which then maintain the correction until fusion of multiple vertebral segments occurs. Surgery may be done from the front (anterior) of the spine or from the back (posterior) of the spine or both, depending on the type and location of the curve.

The treatment goal for soft tissue injury is to restore joint motion and stability to an acceptable functional level. A wide range of treatment options including surgical intervention may be used depending on clinical factors. Surgical treatment involves the repair or replacement of soft tissue elements with autologous or allogenic grafting materials fixed with screws, anchors or through biologic means. Surgery may be performed using open, minimally invasive, or arthroscopic methods. The surgical site and method are highly dependent on the location and extent of injury.

Overall, in addition to external bracing techniques, various surgical techniques are practiced to fuse the instrumented spinal segments. Some of the disadvantages and shortcomings of the surgery may include: poor or slow fusion rate; loss of segmental flexibility; loss of vertebral body height in the skeletally immature patients; poor self-image in adolescent patients who are braced for scoliosis. Lack of curve stabilization; bracing is only successful in approximately 75% of patients. As a result of multiple fusion surgical procedures for lengthening patients as they grow, a subsequent re-operation is as difficult as the original procedure and may require the removal or disablement of implants once a correction of spinal abnormalities is achieved. A further consequence of multiple surgical operations and relative immobility of the fused spine may include the atrophy of the musculature. Some children and adolescents, small in stature may not be physically able to tolerate the surgery required for a definitive fusion procedure.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. Correction treatments used for positioning and alignment may employ implants, such as vertebral rods, for stabilization of a treated section of a spine. This disclosure describes an improvement over these prior art technologies.

### SUMMARY

Accordingly, a surgical system for stabilizing at least two vertebrae of a spine relative to each other is provided. The system includes a spinal construct having an elongated flexible longitudinal element extending between a first end and a second end. At each end of the elongated longitudinal element there is a fixation element capable of securing the elongated longitudinal element and configured to generate a corrective force sufficient to restore the spine to a desired curvature, shape or to correct a deformity of the spine, such as scoliosis.

In certain embodiments, the elongated flexible longitudinal element comprises a tether having a predetermined length, thickness and size. The tether, in some embodiments, has a polygonal or circular cross-section and can be prepared from material selected from fascia, abdominal peritoneum, tendons, gracilis, iliotibial band, small intestine submucosa, perichondrial tissue, completely demineralized bone, partially demineralized bone, ligament, silk, or a combination thereof.

In some embodiments, the spinal construct can be pre-assembled prior to or during surgery.

In some embodiments, the first fixation element or the second fixation element or both comprise a receiver for an anchor member. The receiver has a proximal end and a distal end opposite the proximal end. The anchor member includes the receiver and a lower rod portion attached to the receiver. The lower rod portion of the anchor member includes a head configured to fit within the proximal end of the receiver and a threaded bone engaging member to penetrate soft or hard tissue. At its distal end, the receiver contains an inner threaded surface configured to receive a screw top member having an outer thread surface for engaging with the inner threaded portion of the receiver so that the receiver can secure and adjust the tension of the tether as appropriate to generate a corrective force sufficient to restore the spine to a desired curvature, shape or to correct a deformity of the spine. For example, the screw top member can be a set screw.

In other embodiments the tether can be pre-attached to the receiver of the spine on a back table to form a tether anchor member receiver assembly that can pop or snap on to the head of an already existing bone screw. This pop-on technology has many advantages including that it minimizes manipulation of the many parts associated with existing complex devices, decreases surgical steps, reduces inventory parts, can be attached to already existing pedicle screws from previous surgery, and at the same time reduces anesthesia time and patient bleeding.

In some embodiments the distal end of the receiver includes an eyelet portion for securing the tether through it. The tethering material can be tied, knotted, stitched, glued, welded, clamped, crimped, or otherwise coupled to the receiver of the anchor or any other element of the anchor. For example, in various embodiments, the tethering material can also be fastened around the lower rod portion of the anchor member.

In various other embodiments the receiver defines a passage located between two arms, which extends proximally from a lower base portion of the receiver to form a U-shaped enclosure. The arms of the receiver can be internally threaded to receive a screw top member, for example, a set screw.

In certain embodiments the U-shaped body of the receiver can include the tethering material secured to it that can be any shape including, C-shape, tulip shape, square, rectangular, oval, circular, crescent, or the like.

In other embodiments the tether is received within the threads of the threaded bone engaging member prior to placing it into the at least two vertebrae.

In certain embodiments the tether is detained by the anchor member pressing against a bone surface on both sides of a hole formed within the at least two vertebrae upon driving the threaded bone engaging member into each vertebrae thereby forming a bone-tether-bone assembly.

In various embodiments the first fixation element or the second fixation element or both comprise a dowel rod that can be wedged into a hole drilled in the at least two vertebrae, the hole containing the tether prior to insertion of the dowel rod. In other embodiments, the dowel rod can have a threaded outer surface for retaining the tether on both sides of the threaded dowel rod upon insertion into the hole drilled into the vertebral bodies.

In other embodiments the spinal construct described in this application includes an agent selected from biologically active agents, radiolucent material, radiomarkers, therapeutic agents, pharmacological agents or a combination thereof.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 illustrates a diagrammatic front view of two vertebrae, V1 and V2;

FIG. 2 illustrates a diagrammatic front view of two vertebrae, V1 and V2 each containing part of an anchor member in accordance with an embodiment of the present disclosure;

FIGS. 3, 3A and 3B illustrate a diagrammatic front view of a pre-assembled spinal construct in accordance with another embodiment of the present disclosure;

FIG. 4 illustrates a diagrammatic front view of a spinal system for correction of a spinal disorder in accordance with an embodiment of the present disclosure;

FIG. 5 illustrates a perspective view of an anchor member in accordance with an embodiment of the present disclosure;

FIG. 6 illustrates a diagrammatic front view of a spinal construct in accordance with another embodiment of the present disclosure;

FIG. 7 illustrates a diagrammatic front view of a pre-assembled spinal construct in accordance with yet another embodiment of the present disclosure;

FIG. 8 illustrates a pictorial perspective view of an anchor member having a C-shaped receiver in accordance with another embodiment of the present disclosure;

FIG. 9 illustrates a perspective view of a bone anchor having a tulip shaped receiver in accordance with another embodiment of the present disclosure;

FIG. 10 illustrates a diagrammatic front view of a spinal system for correction of a spinal disorder in accordance with an embodiment of the present disclosure wherein the fixation elements are dowels;

FIG. 11 illustrates a diagrammatic front view of a spinal system for correction of a spinal disorder in accordance with an embodiment of the present disclosure wherein the fixation elements are threaded dowels;

FIG. 12 illustrates a diagrammatic front view of a spinal system for correction of a spinal disorder including an anchor that is a pre-assembled bone-tether-bone sandwich in accordance with an embodiment of the present disclosure;

FIG. 13 illustrates a diagrammatic front view of a spinal system for correction of a spinal disorder including a fixation element wherein the tether material is wrapped within the threaded bone engaging member in accordance with an embodiment of the present disclosure.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

The exemplary embodiments of the spinal construct disclosed are discussed in terms of medical devices for the treatment of musculoskeletal disorders and more particularly, in terms of a spinal construct for fusionless correction of a spine disorder. It is envisioned that the spinal construct may be employed in applications such as fusionless correction of deformities, such as scoliosis. For example, the spinal construct can include attachment of a tether to a convex side of a spine that is curved due to a deformity (e.g., scoliosis). It is contemplated that while the tether may be affixed to a first side of each of a plurality of vertebrae to prevent growth of vertebrae of the first side, the system allows for growth and adjustments to a second side of the plurality of vertebrae.

It is also contemplated that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed surgical system may be alternatively employed in a surgical treatment with a patient in a prone or supine position, and/or employs various surgical approaches to the spine, including anterior, posterior, posterior mid-line, direct lateral, postero-lateral, and/or antero-lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description of the disclosure presented in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” “comprises”, and the like are open ended terms that indicate the presence of stated elements or features, but do not preclude additional elements or features. The articles “a”, “an” and “the” are intended to include the plural as well as the singular, unless the context clearly indicates otherwise.

Further, as used in the specification and including the appended claims, “treating” or “treatment” of a disease or condition refers to performing a procedure that may include administering one or more drugs to a patient in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

In one embodiment there is a system for reducing curvature of a spine, the system comprising a spinal construct having an elongated longitudinal element affixed to and extending between a first fixation element and a second fixation element, the first fixation element having a first end configured to engage at least a portion of a first anchor member, and the second fixation element having a second end configured to engage at least a portion of a second anchor member, the first and second anchor members configured to pierce the spine, wherein the elongated longitudinal element is configured to generate a corrective force sufficient to reduce curvature of the spine.

In another embodiment, a spinal system for stabilizing at least two vertebrae of a spine relative to each other is provided. The system includes a spinal construct comprising an elongated flexible longitudinal element such as a tether extending between a first end and a second end. Each end of the elongated element includes a fixation element, for example a bone anchor assembly, and the elongated longitudinal element is configured to generate a corrective force sufficient to restore the spine to a desired curvature, shape or to correct a deformity of the spine.

It is also envisioned that the spinal system described herein provides features along a sagittal plane of a patient whereby the tether is positioned anterior to a pedicle to reduce undesired lordosis. It is further envisioned that the system and method provided features along a coronal plane of a patient whereby the tether is positioned in a lateral orientation relative to a pedicle to provide more correction in the coronal plane.

The components of the spinal system can be fabricated from biologically acceptable materials suitable for medical applications, including metals, synthetic polymers, ceramics and bone material and/or their composites, depending on the particular application and/or preference of a medical practitioner. For example, the components of the spinal system, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of the spinal construct and universal attachment system may have material composites, including the above materials, to achieve various desired characteristics such as strength, rigidity, elasticity, compliance, biomechanical performance, durability and radiolucency or imaging preference. The components of the bone fastener system, individually or collectively, may also be fabricated from a heterogeneous material such as a combination of two or more of the above-described materials.

The spinal construct provided herein allows a surgeon to select a tether, determine its length, pre-assemble the tether with a receiver of an anchor member assembly on a back table to form a tether anchor member receiver assembly and then pop on or snap the assembly onto the head of an already existing head of the bone screw. This pop-on technology minimizes manipulation of the many parts associated with prior art complex devices, decreases potential surgical fiddle time, decreases surgical steps, and reduces inventory parts. The spinal construct of the present application can be attached to already existing pedicle screws from previous surgery and at the same time reduces anesthesia time and patient bleeding. Utilizing a pre-assembled spinal construct saves many surgical steps that would have been required had the spinal construct been assembled in situ element by element. Moreover, based on patient information available prior to surgery, the spinal construct described in this application can be engineered with great precision ordinarily not available during the surgical procedure.

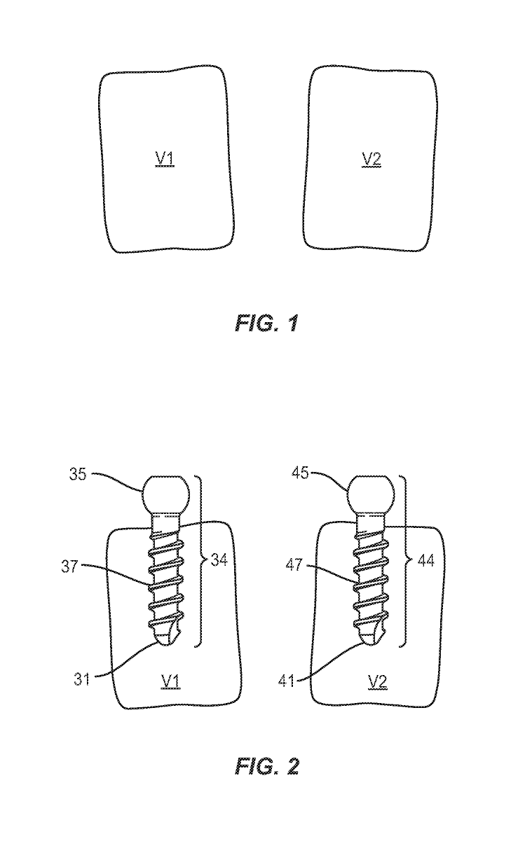

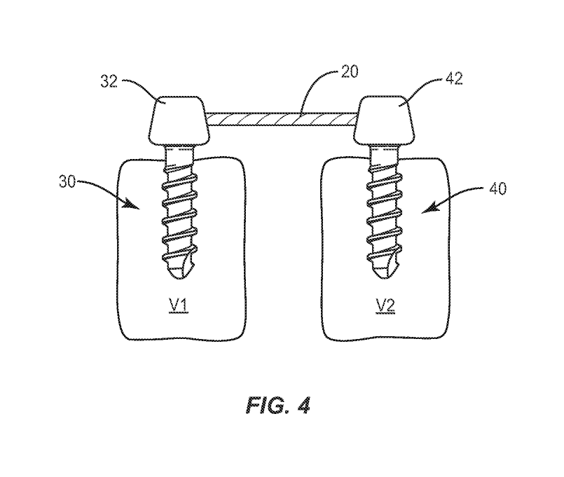

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications in the described devices, instruments, methods, and any further application of the principles of the disclosure as described herein are contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. The following discussion includes a description of a spinal construct and related methods of employing the bone fastener and system in accordance with the principles of the present disclosure. Alternate embodiments are also disclosed. Reference will now be made in detail to the exemplary embodiments of the present disclosure, which are illustrated in the accompanying figures. Turning now to FIGS. 1-13, there are illustrated components of a spinal construct and a universal bone attachment system in accordance with the principles of the present disclosure.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents that may be included within the invention as defined by the appended claims.

As illustrated in FIGS. 1-4, the present system includes a longitudinal element, such as, for example, a tether 20 that extends between a first end 22 and a second end 24. Tether 20 has a flexible configuration, which includes movement in a lateral or side to side direction and prevents expanding and/or extension in an axial direction upon fixation with vertebrae, as will be described. It is envisioned that all or only a portion of tether 20 may have a semi-rigid, rigid or elastic configuration, and/or have elastic properties such that tether 20 provides a selective amount of expansion and/or extension in an axial direction. It is further envisioned that tether 20 may be compressible in an axial direction. Tether 20 can include a plurality of separately attachable or connectable portions or sections, such as bands or loops, or may be monolithically formed as a single continuous element.

Tether 20 has an outer surface 26 and a uniform thickness/diameter. It is envisioned that outer surface 26 may have various surface configurations, such as, for example, rough, threaded for connection with surgical instruments, arcuate, undulating, porous, semi-porous, dimpled, polished and/or textured according to the requirements of a particular application. It is contemplated that the thickness defined by tether 20 may be uniformly increasing or decreasing, or has alternate diameter dimensions along its length. It is further contemplated that tether 20 may have various cross section configurations, such as, for example, oval, oblong, triangular, rectangular, square, polygonal, irregular, uniform, non-uniform, variable and/or tapered.

It is contemplated that tether 20 may have various lengths, according to the requirements of a particular application. It is further contemplated that tether 20 may be braided, such as a rope, or include a plurality elongated elements to provide a predetermined force resistance.

The tethering material may be made from fascia, which, as a term used in this disclosure, describes a single segment, length, piece of tissue capable of maintaining the corrective loads between at least two bony members. As is known, the fascia extends under the skin to cover underlying tissues and to separate different layers of tissue. Accordingly, the flexible material when comprising fascia, can be obtained from the patient's body and, in this case, be characterized as an “autograft” fascia. Alternatively, fascia may be obtained from a foreign body or material and, in this case, be termed as an “allograft” fascia. Structurally, the tethering material can include multiple pieces, bands, or loops, or a single continuous piece or loop.

The tether material may comprise materials other than fascia. Other alternatives include fabrication of the tethering material in whole or in part from biocompatible, non-biodegradable fibers of a native, biosynthetic, or synthetic polymeric, connective tissue or plant connective tissue-like characterized by the biocompatibility of the selected material. The tethering material can comprise non-resorbable, non-biodegradable polymers, metals, similar to a flexible wire or cable. Overall, the tethering material can include abdominal peritoneum, tendons, small intestine submucosa, perichondrial tissue, completely or partially demineralized bone, ligament, silk, or combination thereof. However, it is contemplated that the tether material utilized in this application does not include any biodegradable material.

It is contemplated that the longitudinal element may include one or a plurality of flexible wires, staples, cables, ribbons, artificial and/or synthetic strands, rods, plates, springs, and combinations thereof. In one embodiment, the longitudinal element is a cadaver tendon. In one embodiment, the longitudinal element is a solid core. In one embodiment, the longitudinal element is tubular.

With further reference to FIGS. 1-4, the system for stabilizing at least two vertebrae V1, V2 of a spine relative to each other includes a spinal construct including an elongated flexible longitudinal element, such a tether 20 extending between a first end 22 having a first fixation element, and a second end 24 having a second fixation element. The first fixation element or the second fixation element or both comprise the receiver(s) 32, 42 of an anchor member(s) 30, 40. Anchor member(s) 30, 40 include the receiver(s) 32, 42 and a lower rod portion(s) 34, 44 attached to the receiver(s) 32, 42. Receiver(s) 32, 42 have proximal end(s) 36, 46 and distal end(s) 38, 48 opposite each proximal end. The lower rod portion of the anchor member(s) 30, 40 include a head 35, 45 and threaded bone engaging member(s) 37, 47. The head 35, 45 is configured to fit within the proximal end(s) 36, 46 of the receiver(s) 32, 42. Receiver(s) 32, 42 have an inner threaded surface at distal end(s) 38, 48 and screw top member(s) 39, 49 (not shown) having an outer thread surface for engaging with the inner threaded portion of receiver(s) 32, 42 at the distal end. In this way, by engaging the inner threaded portion of the receiver(s) 32, 42 the screw top member(s) 39, 49 not only retains the tether in the receiver(s) but can also adjust the tension in the tether as appropriate for a given patient.

Tether 20 and receiver(s) 32, 42 form a spinal construct that a surgeon can pre-assemble on the back table prior to the surgical procedure. Subsequently, the assembled spinal construct can be snapped or popped onto previously implanted lower rod portion(s) 34, 44 of the anchor member(s) 30, 40. This pop-on technology minimizes manipulation of the many parts associated with prior art complex devices, decreases surgical steps, and reduces inventory parts. The device of the current application can be attached to already existing pedicle screws from previous surgery and at the same time reduces anesthesia time and patient bleeding.

Various types of anchors can be used to couple tether 20 to the vertebrae V1 and V2 of the spine. In one embodiment, anchors 30, 40 include threaded bone engaging member(s) 37, 47 extending distally from receiver(s) 32, 42. The bone engaging member(s) 37, 47 are sized to extend into vertebrae V1 and V2 and can be threaded or can be in the form of hook or other suitable bone engaging structure. Each bone engaging member 37, 47 include a distal tip(s) 31, 41 and proximal head(s) 35, 45.

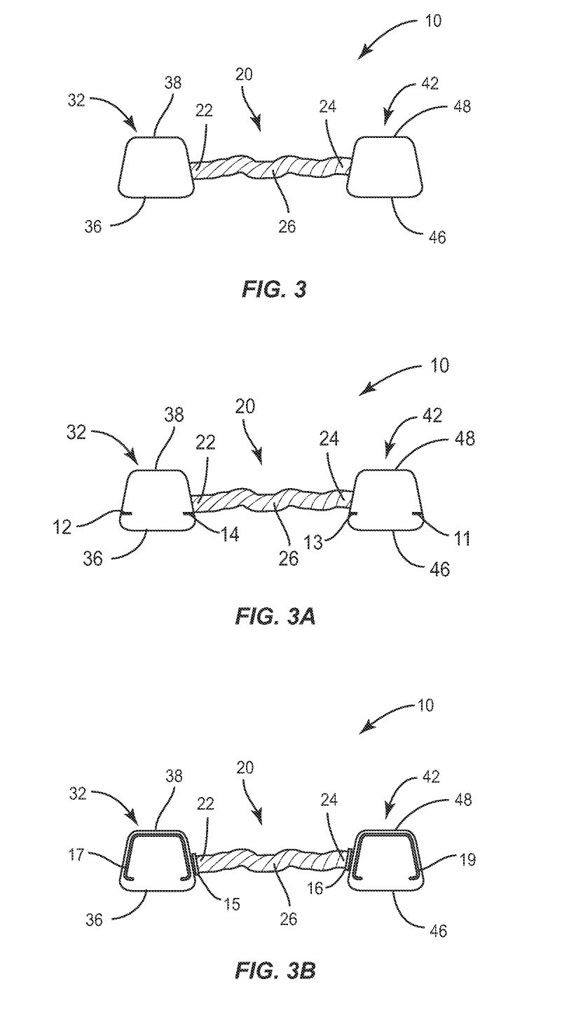

In some embodiments shown in FIG. 3A, the first fixation element comprises a first interference fitting 12 and 14 and the second fixation element comprises a second interference fitting 11 and 13 that are configured to receive a portion of the anchoring member (e.g., a head of a bone fastener, screw, rod, etc.) so that upon sufficient pushing force applied to receivers 32 and 42, they will move the interference fittings and cause them to snap or pop on the anchoring member. The interference fittings 12, 14, 11 and 13 are shown as projections. These projections can be disposed at discrete positions on or in the interior surface of receivers 32 and 42 to ease coupling of the system. When the system is applied, the tether 20 will be taut and apply corrective force to the spine to reduce unwanted curvature of it. The receivers and the interference fitting can be monolithic. Alternatively, in some embodiments, the interference fitting can be disposed at discrete positions on or in an interior surface of the receiver.

It will be understood that although the interference fitting are shown as projections, they can be recesses or a combination of projections and recesses that allow any fixation element to be removably or permanently attached to the anchor member.

In some embodiments, the interference fittings 12, 14, 11 and 13 can comprise deformable material to contact and retain at least a portion of the anchor member in position. In some embodiments, the interference fittings 12, 14, 11 and 13 can comprise expandable material to contact and retain at least a portion of the anchor member in position as it expands. In some embodiments, receivers 32 and 42 have a channel having a diameter that is the same size or smaller than the portion of the anchor that it engages so that upon sufficient pushing force applied to receivers 32 and 42, they will slide over and lock onto the anchoring member.

In some embodiments shown in FIG. 3B, the first fixation element comprises a first interference fitting 17 and the second fixation element comprises a second interference fitting 19 that is configured to receive a portion of the anchoring member (e.g., a head of a bone fastener, screw, rod, etc.) so that upon sufficient pushing force applied to receivers 32 and 42, they will move the interference fittings and cause them to snap or pop on the anchoring member. When the system is applied to the spine, the tether 20 will be taut and apply corrective force to the spine to reduce unwanted curvature of it. Interference fittings 17 and 19 as shown can be disposed on an interior surface of at least the first and/or second fixation element to contact and hold at least a portion of the anchor member in position. In the embodiment shown, the tether 22 can be attached to one or more receivers by reinforcement elements 15 and 16 that reinforce the receivers 32 and 42, when the tether is pulled taught. The reinforcement members can be disposed at the first end 22 and/or the second end 24 of the tether 20. The reinforcement members and the interference fitting can be made of deformable or expandable material.

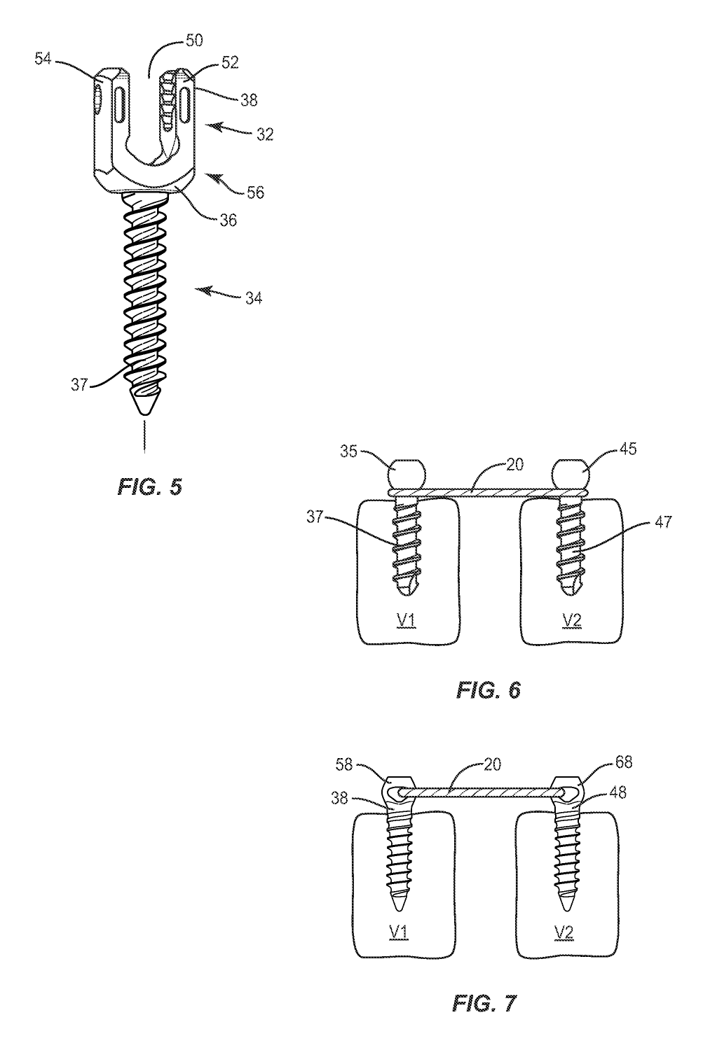

As illustrated in FIG. 5 and with respect to V1 or V2 or both, receiver(s) 32 can define passage(s) 50 for receiving tether 20 therein. Passage 50 is located between first and second arms 52, 54, which extend proximally from a lower base portion 56. Passage 50, can define a U-shape or any other suitable shape. Arms 52, 54 can be internally threaded to threadingly receive screw top member 39 (shown in FIG. 9).

Screw top member(s) 39 can include a proximal tool engaging portion and a distal shaft portion. The shaft portion can be in the form of a set screw to engage arms 52, 54. Other forms for the screw top member 39 are contemplated, including nuts, caps, plugs, and sliding locking elements, for example. In the illustrated embodiment, screw top member 39 can be threaded into passage 50 and into contact with tether 20 to secure it into receiver(s) 32, 42. The tether can contact the opposite surface of 38 and be positioned above 36.

In another embodiment, tether 20 can be stitched around the lower rod portion(s) 34, 44 (of FIG. 2) at a location, for example, between head(s) 35, 45 and threaded bone engaging member(s) 37, 47 as illustrated in FIG. 6.

In another embodiment receiver(s) 32, 42 of FIGS. 2 and 3 can include an eyelet portion(s) 58, 68 attached directly to the distal end 38, 48 of the receiver as illustrated in FIG. 7. The eyelet portion is configured so that the tether 20 can be tied, knotted, glued, welded, clamped, crimped, or otherwise coupled to the anchor.

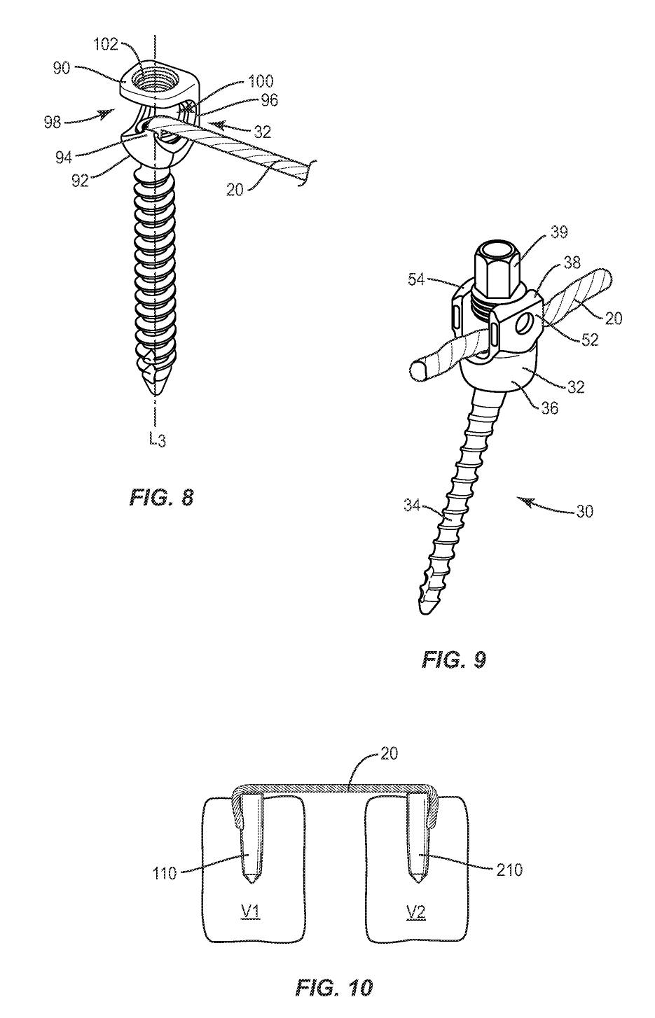

In various embodiments, the body of receiver(s) 32 can be any shape including, U-shaped, C-shaped, tulip shaped, square, rectangular, oval, circular, or the like. For example, in certain embodiments as illustrated in FIG. 8, receiver 32 is substantially C-shaped, having an upper leg 90, a lower leg 92 including foot portion 94 extending from one end thereof, and an intermediate portion 96 joining upper and lower legs 90, 92 opposite of foot portion 94. Receiver 32 defines a mouth 98 between upper leg 90 and foot portion 94 that is opposite intermediate portion 96. Mouth 98 opens into upper passage portion 100 extending through receiver 32, with upper passage portion 100 extending in an orthogonal relationship to longitudinal axis L₃. Upper leg 90 has a threaded aperture 102 into which an engagement member or set screw 39 can be threadingly engaged to retain tether 20. Either or both ends of tether 20 can be widened or fluted so that tether 20 is securely detained by receiver 32 and unlikely to escape the grip of C-shape receiver 32.

In another embodiment, as illustrated in FIG. 9 a bone anchor 30 comprises a lower rod portion 34 with a tulip shaped top receiver 32. The tulip shaped receiver 32 comprises distal portion 38 and a proximal portion 36 with a first arm 52 and a second arm 54. Together, the first and second arms 52, 54 form a substantially U-shaped passage 50 into which tether 20 may be axially positioned. The tulip shaped distal portion 38 of receiver 32 has an inner threaded surface configured to accept a set screw 39, which detains tether 20 within the U-shaped passage by pinching or piercing it in place.

In other embodiments, the first fixation element or the second fixation element or both comprise a dowel rod(s) 110, 210 as illustrated in FIG. 10. Dowel rod(s) 110, 210 are a solid cylindrical rod made of allograft bone that can be wedged in holes previously drilled into at least vertebrae V1 and V2 to provide means for securing tether 20 onto vertebrae V1 and V2.

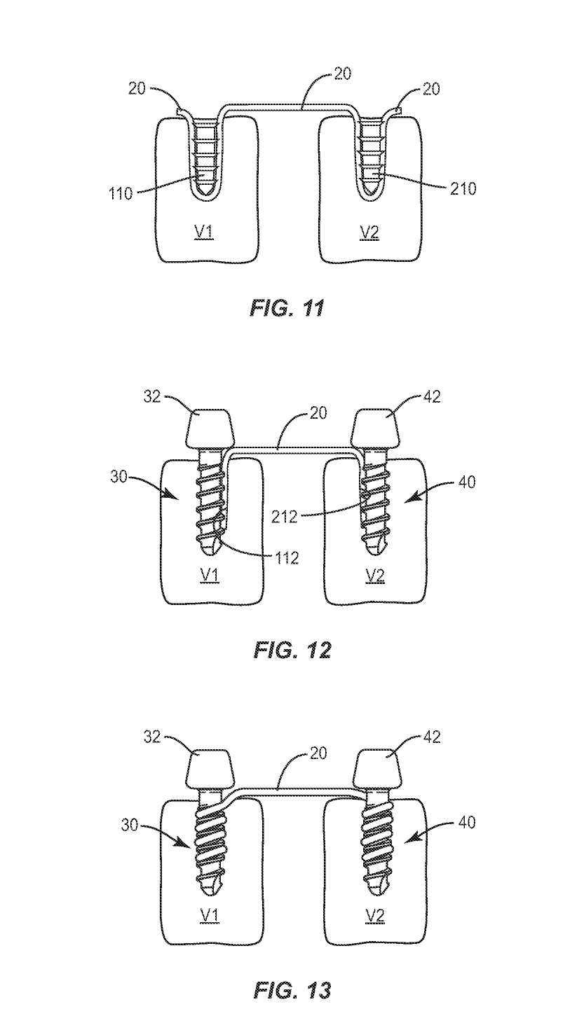

In other aspects of this application, dowel rod(s) 110, 210 have an outer threaded surface for engaging into previously drilled holes into at least vertebrae V1 and V2. Tether 20 is secured by each threaded dowel by pressing the tether towards the outer surface of holes on both sides of each dowel in an interference technique as illustrated in FIG. 11.

In yet another embodiment, tether 20 and the receiver(s) 32, 42 can form a pre-assembled spinal construct that is a bone-tether-bone sandwich as illustrated in FIG. 12. In FIG. 12, tether 20 is pressed between a surface(s) 112 and 212 formed within vertebral bodies V1 and V2 upon driving threaded bone engaging member(s) 37, 47 into each vertebrae V1 and V2. Receiver(s) 32, 42 of anchor(s) 30, 40 press tether 20 against outer surface(s) 112, 212 of vertebral bodies V1 and V2 thereby detaining the tether in a tight bone-tether-bone sandwich.

In a related embodiment illustrated in FIG. 13, tether 20 can be wrapped within the threads of the anchors 30, 40 as shown before the anchors is placed into pre-drilled holes in vertebrae V1 and V2. The receivers 32 and 42 can be pressed or snapped on to the head of fastener 30 and 40. In some embodiments, the receivers can have a friction fitting or a push fitting that eases coupling of the receivers to the head of the fastener.

In one embodiment, the fusionless correction system described above includes an agent, which may be disposed, packed or layered within, on or about the components and/or surfaces of the fusionless correction system. It is envisioned that the agent may include bone growth promoting material, such as, for example, a bone graft or growth factor to enhance fixation of the fixation elements with vertebrae V1 and V2.

Growth factors that can be used with the fusionless correction system include osteoinductive agents (e.g., agents that cause new bone growth in an area where there was none) and/or osteoconductive agents (e.g., agents that cause ingrowth of cells into and/or through a matrix). Osteoinductive agents can be polypeptides or polynucleotides compositions. Polynucleotide compositions of the osteoinductive agents include, but are not limited to, isolated Bone Morphogenic Protein (BMP), Vascular Endothelial Growth Factor (VEGF), Connective Tissue Growth Factor (CTGF), Osteoprotegerin, Growth Differentiation Factors (GDFs), Cartilage Derived Morphogenic Proteins (CDMPs), Lim Mineralization Proteins (LMPs), Platelet derived growth factor, (PDGF or rhPDGF), Insulin-like growth factor (IGF) or Transforming Growth Factor beta (TGF-beta) polynucleotides. Polynucleotide compositions of the osteoinductive agents include, but are not limited to, gene therapy vectors harboring polynucleotides encoding the osteoinductive polypeptide of interest. Gene therapy methods often utilize a polynucleotide, which codes for the osteoinductive polypeptide operatively linked or associated to a promoter or any other genetic elements necessary for the expression of the osteoinductive polypeptide by the target tissue. Such gene therapy and delivery techniques are known in the art (see, for example, International Publication No. WO90/11092, the disclosure of which is herein incorporated by reference in its entirety). Suitable gene therapy vectors include, but are not limited to, gene therapy vectors that do not integrate into the host genome. Alternatively, suitable gene therapy vectors include, but are not limited to, gene therapy vectors that integrate into the host genome.

In some embodiments, the polynucleotide is delivered in plasmid formulations. Plasmid DNA or RNA formulations refer to polynucleotide sequences encoding osteoinductive polypeptides that are free from any delivery vehicle that acts to assist, promote or facilitate entry into the cell, including viral sequences, viral particles, liposome formulations, lipofectin, precipitating agents or the like. Optionally, gene therapy compositions can be delivered in liposome formulations and lipofectin formulations, which can be prepared by methods well known to those skilled in the art. General methods are described, for example, in U.S. Pat. Nos. 5,593,972, 5,589,466, and 5,580,859, the disclosures of which are herein incorporated by reference in their entireties.

Gene therapy vectors further comprise suitable adenoviral vectors including, but not limited to for example, those described in U.S. Pat. No. 5,652,224, which is herein incorporated by reference.

Polypeptide compositions of the isolated osteoinductive agents include, but are not limited to, isolated Bone Morphogenic Protein (BMP), Vascular Endothelial Growth Factor (VEGF), Connective Tissue Growth Factor (CTGF), Osteoprotegerin, Growth Differentiation Factors (GDFs), Cartilage Derived Morphogenic Proteins (CDMPs), Lim Mineralization Proteins (LMPs), Platelet derived growth factor, (PDGF or rhPDGF), Insulin-like growth factor (IGF) or Transforming Growth Factor beta (TGF-beta707) polypeptides. Polypeptide compositions of the osteoinductive agents include, but are not limited to, full length proteins, fragments or variants thereof.

Variants of the isolated osteoinductive agents include, but are not limited to, polypeptide variants that are designed to increase the duration of activity of the osteoinductive agent in vivo. Typically, variant osteoinductive agents include, but are not limited to, full length proteins or fragments thereof that are conjugated to polyethylene glycol (PEG) moieties to increase their half-life in vivo (also known as pegylation). Methods of pegylating polypeptides are well known in the art (See, e.g., U.S. Pat. No. 6,552,170 and European Pat. No. 0,401,384 as examples of methods of generating pegylated polypeptides). In some embodiments, the isolated osteoinductive agent(s) are provided as fusion proteins. In one embodiment, the osteoinductive agent(s) are available as fusion proteins with the Fc portion of human IgG. In another embodiment, the osteoinductive agent(s) are available as hetero- or homodimers or multimers. Examples of some fusion proteins include, but are not limited to, ligand fusions between mature osteoinductive polypeptides and the Fc portion of human Immunoglobulin G (IgG). Methods of making fusion proteins and constructs encoding the same are well known in the art.

Isolated osteoinductive agents that can be used with the fusionless correction system described above are typically sterile. In a non-limiting method, sterility is readily accomplished for example by filtration through sterile filtration membranes (e.g., 0.2 micron membranes or filters). In one embodiment, the isolated osteoinductive agents include one or more members of the family of Bone Morphogenic Proteins (“BMPs”). BMPs are a class of proteins thought to have osteoinductive or growth-promoting activities on endogenous bone tissue, or function as pro-collagen precursors. Known members of the BMP family include, but are not limited to, BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, BMP-18 as well as polynucleotides or polypeptides thereof, as well as mature polypeptides or polynucleotides encoding the same.

BMPs utilized as osteoinductive agents comprise one or more of BMP-1; BMP-2; BMP-3; BMP-4; BMP-5; BMP-6; BMP-7; BMP-8; BMP-9; BMP-10; BMP-11; BMP-12; BMP-13; BMP-15; BMP-16; BMP-17; or BMP-18; as well as any combination of one or more of these BMPs, including full length BMPs or fragments thereof, or combinations thereof, either as polypeptides or polynucleotides encoding the polypeptide fragments of all of the recited BMPs. The isolated BMP osteoinductive agents may be administered as polynucleotides, polypeptides, full length protein or combinations thereof.

In another embodiment, isolated osteoinductive agents include osteoclastogenesis inhibitors to inhibit bone resorption of the bone tissue surrounding the site of implantation by osteoclasts. Osteoclast and osteoclastogenesis inhibitors include, but are not limited to, osteoprotegerin polynucleotides or polypeptides, as well as mature osteoprotegerin proteins, polypeptides or polynucleotides encoding the same. Osteoprotegerin is a member of the TNF-receptor superfamily and is an osteoblast-secreted decoy receptor that functions as a negative regulator of bone resorption. This protein specifically binds to its ligand, osteoprotegerin ligand (TNFSF11/OPGL), both of which are key extracellular regulators of osteoclast development.

Osteoclastogenesis inhibitors further include, but are not limited to, chemical compounds such as bisphosphonate, 5-lipoxygenase inhibitors such as those described in U.S. Pat. Nos. 5,534,524 and 6,455,541 (the contents of which are herein incorporated by reference in their entireties), heterocyclic compounds such as those described in U.S. Pat. No. 5,658,935 (herein incorporated by reference in its entirety), 2,4-dioxoimidazolidine and imidazolidine derivative compounds such as those described in U.S. Pat. Nos. 5,397,796 and 5,554,594 (the contents of which are herein incorporated by reference in their entireties), sulfonamide derivatives such as those described in U.S. Pat. No. 6,313,119 (herein incorporated by reference in its entirety), or acylguanidine compounds such as those described in U.S. Pat. No. 6,492,356 (herein incorporated by reference in its entirety).

In another embodiment, isolated osteoinductive agents include one or more members of the family of Connective Tissue Growth Factors (“CTGFs”). CTGFs are a class of proteins thought to have growth-promoting activities on connective tissues. Known members of the CTGF family include, but are not limited to, CTGF-1, CTGF-2, CTGF-4 polynucleotides or polypeptides thereof, as well as mature proteins, polypeptides or polynucleotides encoding the same.

In another embodiment, isolated osteoinductive agents include one or more members of the family of Vascular Endothelial Growth Factors (“VEGFs”). VEGFs are a class of proteins thought to have growth-promoting activities on vascular tissues. Known members of the VEGF family include, but are not limited to, VEGF-A, VEGF-B, VEGF-C, VEGF-D, VEGF-E or polynucleotides or polypeptides thereof, as well as mature VEGF-A, proteins, polypeptides or polynucleotides encoding the same.

In another embodiment, isolated osteoinductive agents include one or more members of the family of Transforming Growth Factor-beta (“TGFbetas”). TGF-betas are a class of proteins thought to have growth-promoting activities on a range of tissues, including connective tissues. Known members of the TGF-beta family include, but are not limited to, TGF-beta-1, TGF-beta-2, TGF-beta-3, polynucleotides or polypeptides thereof, as well as mature protein, polypeptides or polynucleotides encoding the same.

In another embodiment, isolated osteoinductive agents include one or more Growth Differentiation Factors (“GDFs”). Known GDFs include, but are not limited to, GDF-1, GDF-2, GDF-3, GDF-7, GDF-10, GDF-11, and GDF-15. For example, GDFs useful as isolated osteoinductive agents include, but are not limited to, the following GDFs: GDF-1 polynucleotides or polypeptides corresponding to GenBank Accession Numbers M62302, AAA58501, and AAB94786, as well as mature GDF-1 polypeptides or polynucleotides encoding the same. GDF-2 polynucleotides or polypeptides corresponding to GenBank Accession Numbers BC069643, BC074921, Q9UK05, AAH69643, or AAH74921, as well as mature GDF-2 polypeptides or polynucleotides encoding the same. GDF-3 polynucleotides or polypeptides corresponding to GenBank Accession Numbers AF263538, BC030959, AAF91389, AAQ89234, or Q9NR23, as well as mature GDF-3 polypeptides or polynucleotides encoding the same. GDF-7 polynucleotides or polypeptides corresponding to GenBank Accession Numbers AB158468, AF522369, AAP97720, or Q7Z4P5, as well as mature GDF-7 polypeptides or polynucleotides encoding the same. GDF-10 polynucleotides or polypeptides corresponding to GenBank Accession Numbers BC028237 or AAH28237, as well as mature GDF-10 polypeptides or polynucleotides encoding the same.

GDF-11 polynucleotides or polypeptides corresponding to GenBank Accession Numbers AF100907, NP_{—}005802 or 095390, as well as mature GDF-11 polypeptides or polynucleotides encoding the same. GDF-15 polynucleotides or polypeptides corresponding to GenBank Accession Numbers BC008962, BC000529, AAH00529, or NP 004855, as well as mature GDF-15 polypeptides or polynucleotides encoding the same.

In another embodiment, isolated osteoinductive agents include Cartilage Derived Morphogenic Protein (CDMP) and Lim Mineralization Protein (LMP) polynucleotides or polypeptides. Known CDMPs and LMPs include, but are not limited to, CDMP-1, CDMP-2, LMP-1, LMP-2, or LMP-3.

CDMPs and LMPs useful as isolated osteoinductive agents include, but are not limited to, the following CDMPs and LMPs: CDMP-1 polynucleotides and polypeptides corresponding to GenBank Accession Numbers NM_{—}000557, U13660, NP_{—}000548 or P43026, as well as mature CDMP-1 polypeptides or polynucleotides encoding the same. CDMP-2 polypeptides corresponding to GenBank Accession Numbers or P55106, as well as mature CDMP-2 polypeptides. LMP-1 polynucleotides or polypeptides corresponding to GenBank Accession Numbers AF345904 or AAK30567, as well as mature LMP-1 polypeptides or polynucleotides encoding the same. LMP-2 polynucleotides or polypeptides corresponding to GenBank Accession Numbers AF345905 or AAK30568, as well as mature LMP-2 polypeptides or polynucleotides encoding the same. LMP-3 polynucleotides or polypeptides corresponding to GenBank Accession Numbers AF345906 or AAK30569, as well as mature LMP-3 polypeptides or polynucleotides encoding the same.

In another embodiment, isolated osteoinductive agents include one or more members of any one of the families of Bone Morphogenic Proteins (BMPs), Connective Tissue Growth Factors (CTGFs), Vascular Endothelial Growth Factors (VEGFs), Osteoprotegerin or any of the other osteoclastogenesis inhibitors, Growth Differentiation Factors (GDFs), Cartilage Derived Morphogenic Proteins (CDMPs), Lim Mineralization Proteins (LMPs), or Transforming Growth Factor-betas (TGF-betas), as well as mixtures or combinations thereof.

In another embodiment, the one or more isolated osteoinductive agents useful in conjunction with the spinal system are selected from the group consisting of BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, BMP-18, or any combination thereof; CTGF-1, CTGF-2, CGTF-3, CTGF-4, or any combination thereof; VEGF-A, VEGF-B, VEGF-C, VEGF-D, VEGF-E, or any combination thereof; GDF-1, GDF-2, GDF-3, GDF-7, GDF-10, GDF-11, GDF-15, or any combination thereof CDMP-1, CDMP-2, LMP-1, LMP-2, LMP-3, and/or any combination thereof. Osteoprotegerin; TGF-beta-1, TGF-beta-2, TGF-beta-3, or any combination thereof or any combination of one or more members of these groups.

It is contemplated that the agent to be used with the spinal system may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as hydroxyapatite, calcium phosphate and calcium sulfite, biologically active agents, for example, gradual release compositions such as by blending in a bioresorbable polymer that releases the biologically active agent or agents in an appropriate time dependent fashion as the polymer degrades within the patient.

The components of the fusionless correction system can be made of radiolucent materials such as polymers. Radiomarkers may be included for identification under x-ray, fluoroscopy, CT or other imaging techniques. It is envisioned that the agent may include one or a plurality of therapeutic agents and/or pharmacological agents for release, including sustained release, to treat, for example, pain, inflammation and degeneration.

It is contemplated that the components of the fusionless correction system described above may be employed to treat progressive idiopathic scoliosis with or without sagittal deformity in either infantile or juvenile patients, including but not limited to prepubescent children, adolescents from 10-12 years old with continued growth potential, and/or older children whose growth spurt is late or who otherwise retain growth potential. It is further contemplated that the components of the fusionless correction system may be used to prevent or minimize curve progression in individuals of various ages.

In some embodiments, there is a method for reducing curvature of a spine, the method comprising providing a spinal construct having an elongated longitudinal element affixed to and extending between a first fixation element and a second fixation element, the first fixation element having a first end configured to engage at least a portion of a first anchor member, and the second fixation element having a second end configured to engage at least a portion of a second anchor member; affixing the first end of the fixation element to the first anchor member, wherein the anchor member is implanted in a first vertebra and affixing the second end of the second fixation element to a second vertebra so as to cause the elongated longitudinal element to generate a force against the spine sufficient to reduce curvature of the spine.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

1. A system for reducing curvature of a spine, the system comprising a spinal construct having an elongated longitudinal element affixed to and extending between a first fixation element and a second fixation element, the first fixation element having a first end configured to engage at least a portion of a first anchor member, and the second fixation element having a second end configured to engage at least a portion of a second anchor member, the first and second anchor members configured to pierce the spine, the first and/or second fixation element comprises a receiver and the first and second anchor members comprise a rod portion, the rod portion of the anchor member having a head and a bone engaging member, the head configured to fit within the receiver, wherein the elongated longitudinal element is configured to adjust tension between the first and second fixation elements as the first and second anchor members pierce the spine, wherein the elongated longitudinal element is configured to generate a corrective force sufficient to reduce curvature of the spine.

2. A system according to claim 1, wherein the elongated longitudinal element comprises (i) a tether having a predetermined length, thickness, and size; (ii) a flexible tether having a predetermined length, thickness, and size; or (iii) a tether having at least one reinforcement member.

3. A system according to claim 1, wherein the first fixation element or the second fixation element or both comprise an interference fitting configured to retain at least the head of the anchor members.

4. A system according to claim 3, wherein the interference fitting comprises (i) a plurality of projections configured to contact and retain at least a portion of the first or second anchor member in position; (ii) a channel having a diameter smaller or the same size as at least a portion of the anchor members; (iii) a deformable member to contact and retain at least a portion of the anchor members in position; or (iv) an expandable member to retain and hold at least a portion of the anchor members in position.

5. A system according to claim 3, wherein the interference fitting extends on an interior surface of at least the first and/or second fixation element to contact and hold at least a portion of the anchor members in position.

6. A system according to claim 2, wherein the tether has a polygonal or circular cross-section and is prepared from material selected from fascia, abdominal peritoneum, tendons, gracilis, iliotibial band, small intestine submucosa, perichondrial tissue, completely demineralized bone, partially demineralized bone, ligament, silk, or a combination thereof.

7. A system according to claim 2, wherein the tether has a polygonal or circular cross-section and is prepared from material selected from stainless steel alloys, commercially pure titanium, titanium alloys, Grade 5 titanium, super-elastic titanium alloys, cobalt-chrome alloys, stainless steel alloys, superelastic metallic alloys, ceramics, calcium phosphate, polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid or rigid materials, elastomers, rubbers, thermoplastic elastomers, thermoset elastomers, elastomeric composites, polyphenylene, polyamide, polyimide, polyetherimide, polyethylene, epoxy, bone material including autograft, allograft, xenograft or transgenic cortical and/or corticocancellous bone, tissue growth factor, differentiation factors, composites of metals or calcium-based ceramics, composites of PEEK, calcium based ceramics, composites of PEEK with resorbable polymers, or combinations thereof.

8. A system for reducing curvature of a spine, the system comprising a spinal construct having an elongated longitudinal element affixed to and extending between a first fixation element and a second fixation element, the first fixation element having a first end configured to engage at least a portion of a first anchor member, and the second fixation element having a second end configured to engage at least a portion of a second anchor member, the first and second anchor members configured to pierce the spine, wherein the elongated longitudinal element is configured to generate a corrective force sufficient to reduce curvature of the spine, wherein the first and/or second fixation element comprises a receiver and the first and second anchor members comprise a rod portion, the receiver having a proximal end and a distal end opposite the proximal end, the rod portion of the anchor member having a head and a threaded bone engaging member, the head configured to fit within the proximal end of the receiver, the receiver configured to receive the tether and having an inner threaded surface at the distal end for receiving a screw top member, the screw top member configured to allow fastening of the tether between the screw top member and the receiver.

9. A system according to claim 8, wherein the tether is pre-attached to the receiver.

10. A system according to claim 8, wherein the distal end of the receiver comprises an eyelet for threading the tether there through.

11. A system according to claim 10, wherein the tether is fastened around the eyelet.

12. A system according to claim 8, wherein the tether is fastened around the lower rod portion of the anchor.

13. A system according to claim 8, wherein the receiver extends along a longitudinal axis between a proximal end portion and a distal end portion, the receiver comprising an upper leg and a lower leg opposite the upper leg, the lower leg including a foot portion extending from one end thereof, the upper leg extending transversely to the longitudinal axis; an intermediate portion opposite the foot portion, the intermediate portion extending between the upper leg and the lower leg, the lower leg and the intermediate portion defining a cavity configured to receive and retain the tether of the spinal construct.

14. A system according to claim 13, wherein the first end or the second end or both ends of the tether are widened or fluted for retention by the receiver.

15. A system according to claim 8, wherein the receiver is tulip shaped and comprises a first arm and a second arm, the first and second arms forming a substantially U-shaped passage into which the tether is retained by pinching or piercing it in place with the screw top member.

16. A system according to claim 8, wherein the tether is retained by the anchor member pressing against a bone surface on both sides of a hole formed within the at least two vertebrae upon driving the threaded bone engaging member into each vertebrae thereby forming a bone-tether-bone assembly.

17. A system according to claim 1, wherein the first and/or second anchor member comprises a dowel rod wedged into a hole drilled in the at least two vertebrae, the hole containing the tether prior to insertion of the dowel rod.

18. A method for reducing curvature of a spine, the method comprising providing a spinal construct having an elongated longitudinal element affixed to and extending between a first fixation element and a second fixation element, the first and second fixation elements each comprising a receiver, a first anchor member and a second anchor member each comprising a rod portion, the rod portion of the anchor members having a head and a bone engaging member, the heads configured to fit within the receivers, wherein the elongated longitudinal element is configured to adjust tension between the first and second fixation elements as the first and second anchor members pierce the spine; affixing the receiver of the first fixation element to the head of the first anchor member, wherein the bone engaging member of the first anchor member is implanted in a first vertebra and affixing the receiver of the second fixation element to the head of the second anchor member, wherein the bone engaging member of the second anchor member is implanted in a second vertebra so as to cause the elongated longitudinal element to generate a force against the spine sufficient to reduce curvature of the spine.

19. A method for reducing curvature of a spine according to claim 18, wherein the first fixation element or the second fixation element or both comprise an interference fitting configured to retain at least the head of the anchor members upon application of force to the first or second fixation element.

20. A method for reducing curvature of a spine according to claim 19, wherein the interference fitting comprises (i) a plurality of projections configured to contact and retain at least a portion of the anchor members in position upon application of a pushing force to the first or second fixation element; (ii) a channel having a diameter smaller or the same size as at least a portion of the anchor members; (iii) a deformable member to contact and retain at least a portion of the anchor members in position; or (iv) an expandable member to retain and hold at least a portion of the anchor members in position.
